(19) United States
(12) Patent Application Publication
(10) Pub. No.: US 2015/0209307 A1
(43) Pub. Date: Jul. 30, 2015

(54) COMPOSITIONS AND METHODS FOR TREATING MOOD DISORDERS OR SKIN DISEASE OR DAMAGE

(71) Applicant: Eric Hauser KUHRTS, Novato, CA (US)

(72) Inventor: Eric Hauser Kuhrts, Novato, CA (US)

(21) Appl. No.: 14/420,622

(22) PCT Filed: Aug. 8, 2013
(86) PCT No.: PCT/US2013/054120

§ 371 Date: Feb. 9, 2015

Related U.S. Application Data
(60) Provisional application No. 61/682,100, filed on Aug. 10, 2012.

(51) Int. Cl. A61K 31/122; A23L 2/52; A61K 9/00; A61K 47/44; A61K 36/185

(52) U.S. Cl. CPC A61K 31/122; A61K 47/44; A61K 36/185; A61K 9/0095; A61K 9/0014; A23L 2/52; A23V 2002/00

(57) ABSTRACT

Methods for the treatment of mood disorders and/or skin disease or damage (eczema, atopic dermatitis, or wrinkles, for example) can comprise administering an extracted prenylflavonoid to a subject experience the mood disorder or the skin disease or damage.

### BACKGROUND

It is well known that impaired neurotransmission, resulting from low neurotransmitter levels and/or decreased neurotransmitter receptor affinity, is related to mental disease, such as depression, generalized anxiety disorder (GAD), and increased susceptibility to stress and cognitive dysfunction. Compounds that increase neurotransmitter levels in the brain and thus enhance their transmission can exhibit antidepressant properties and exert beneficial effects on a variety of other mental disorders. The main neurotransmitters are serotonin, dopamine, noradrenaline (norepinephrine), acetylcholine, glutamate and gamma-aminobutyric acid (GABA). Neurotransmitters of particular relevance to mood-related disorders include serotonin, noradrenaline, and dopamine, while glutamate and acetylcholine neurotransmission are involved in cognitive function. Enhanced or prolonged neurotransmission is achieved by increasing the concentration of the neurotransmitter in the synaptic cleft, through inhibition of re-uptake into the pre-synaptic nerve ending, or by preventing neurotransmitter catabolism by inhibition of degrading enzymes, such as monoamine oxidase (MAO)-A and -B. Also of interest are the neuropeptides, such as calcitonin gene-related peptide (CGRP).

Commonly prescribed tricyclic antidepressants (TCA), such as imipramine, amitriptyline, and clomipramine, act by inhibiting the re-uptake of serotonin and noradrenaline. These drugs are widely regarded as among the most effective antidepressants available, but unfortunately have a number of disadvantages because they additionally interact with muscarinic, acetylcholine, histamine, and serotonin receptors. Side effects resulting from the use of these drugs include dry mouth, blurred vision, constipation and urinary retention, in addition to postural hypotension. Also, TCAs are not safe when taken in overdose, frequently showing acute cardiotoxicity and other possible side-effects.

Another class of antidepressant drugs is selective serotonin re-uptake inhibitors (SSRI). This class of drugs includes fluoxetine, paroxetine, sertraline, citalopram and fluvoxamine, and act by blocking the serotonin transporter (SERT), a high affinity sodium chloride-dependent neurotransmitter transporter that terminates serotonergic neurotransmission by re-uptake of serotonin. These drugs have been proven effective in the treatment of depression and anxiety as TCAs, and are usually better tolerated. These medications are typically started at low dosages and are increased until they reach a therapeutic level. A common side effect is nausea. Other possible side effects include decreased appetite, dry mouth, sweating, infection, constipation, tremor, yawning, sleepiness and sexual dysfunction.

In addition, compounds that prevent the catabolism of neurotransmitters more broadly by inhibiting MAOs-A and -B exhibit antidepressant effects. MAOs work by catalyzing the oxidation of amine group-containing neurotransmitters such as serotonin, noradrenaline and dopamine.

There is a need for therapeutic compounds for the treatment or prevention of mental diseases and/or disorders which do not show the negative side effects of known antidepressants. Patients with mood disorders are interested in alternative therapies which could minimize the side effects associated with high doses of drugs and yield additional clinical benefits. Severe depression is a long-lasting and recurring disease, which is usually poorly diagnosed. Mild depression is a much more common problem in modern society, and many more patients suffer from mild or moderately severe depression. Thus, there is an increasing interest in the development of therapeutic compounds, as well as pharmaceutical and/or dietary compositions, which may be used to treat or prevent mental diseases/disorders such as depression and dysthymia, in people at risk, to stabilize mood and achieve emotional balance.

Mood disorders, emotional imbalance, and occupational stress can lead to sleep disorders, insomnia, low sleep quality and general disturbances in circadian rhythms (so-called biorhythms), and such conditions can be chronic and persistent in nature. Also, deregulation of circadian rhythms induced by long-haul flights (jet-lag) and shift-work can cause similar symptoms and distress. Therefore, treatment with therapeutic supplementation to maintain a normal circadian rhythm and/or to alleviate and prevent symptoms associated with a disturbed circadian rhythm, such as impairment of cognitive function and memory and mental and physical fatigue, and resulting in improving the overall quality of life and benefiting from improved social integration, would be most desirable.

### DETAILED DESCRIPTION

In describing and claiming the present invention, the following terminology will be used.

The singular forms “a,” “an,” and “the” include plural referents unless the context clearly dictates otherwise. Thus, for example, reference to “a non-ionic surfactant” includes reference to one or more of such compounds.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

In the context of the present disclosure, the term “disorder” also encompasses diseases.

As used herein, a “prenylflavonoid,” or prenylflavonoid, refers to a prenylated compound having a substituted or unsubstituted phenol attached to a phenyl via a C3 alkylene substituted with an oxo group. The C3 alkylene may be present in a linear chain arrangement (e.g. a chalcone) or joined with other atoms to form a substituted or unsubstituted ring (e.g. a flavanone). Prenylflavonoids may be derived from natural sources (e.g. hops), or synthesized chemically. A “prenylated” compound refers to those compounds with an attached —CH₂—CH═C(CH₃)₂ group (e.g. geranylated compounds), optionally hydroxylated prenyl tautomers (e.g. —CH₂—CH—C(CH₃)═CH₂, or —CH₂—C(OH)—C(CH₃)═CH₂), and optionally hydroxylated circularized prenyl derivatives having the formula below:

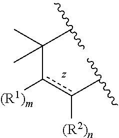

In this formula, the dashed bond z represents a double bond or a single bond. R¹ and R² are independently hydrogen or OH. The symbol [symbol] represents the point of attachment to the remainder of the prenylated compounds.

As used herein, the term “extracted” prenylflavonoid or xanthohumol refers a prenylflavonoid or xanthohumol that has been extracted by any number of extraction methods including ethanol extractions, supercritical carbon dioxide extractions, or the like. The prenylflavonoid, after extraction, will typically be at least 90 wt % pure, but can be 95 wt %, 97 wt %, 98 wt %, or even 99 wt % pure. To reach an effective concentration in the blood to effect a mood disorder or to improve skin health, a threshold concentration can be used to activate or up regulate the oxytocin gene, and thereby oxytocin itself. To reach this effective concentration, a more purified extract of hops can be used. Hops, as it occurs in nature, or even low prenylflavonoid extracts, contain little prenylflavonoids in general and even less xanthohumol. Furthermore, as absorption of the prenylflavonoids are very limited due to low bioavailability, low concentrations are not particularly useful. Therefore, purified extracts containing greater than 20% prenylflavonoid, and even up to 99%, are more effective for activating the oxytocin genes (see concentration in mg/ml necessary to activate the genes in the oxytocin gene example). Thus, even when a final formulation has very little prenylflavonoids because it is diluted by other additives, it is the extracted concentrated prenylflavonoids, such as xanthohumol that is present that provides the therapeutic dose which is superior to naturally occurring concentrations of similar components. In addition, many hops extracts have a very objectionable smell and taste, whereas, extracted and purified prenylflavonoid, including xanthohumol, have a more acceptable smell and pleasant taste. Therefore, a more pure extract is more acceptable for both user compliance, skin care, and to make a pleasant tasting beverage or other consumable.

A “non-ionic surfactant,” as used herein, is a surface active agent that tends to be non-ionized (i.e. uncharged) in neutral solutions (e.g. neutral aqueous solutions).

As used herein, the term “oxytocin” or OT or OXY, shall mean the nine amino acid central nervous system neuropeptide or hormone. Oxytocin is produced in the supraoptic and paraventricular nuclei of the hypothalamus and is mainly released by exocytosis from the neurohypophysis and nerve terminals in response to numerous types of physiological stimuli. The term “oxytocin receptors or OTR” means the receptors which oxytocin binds to in oxytocin cells, triggering an increase in calcium ions. Oxytocin has been called the “hormone of happiness,” and due to its correlation with many positive mental states, could be considered a target for drug therapy for afflictive mood disorders.

As used herein, the oxytocin receptor genes, or OXTR, or OXT mean the genes located on chromosome 3p25.3, that code for the oxytocin receptor, the receptor through which the neurohormone oxytocin exerts a range of effects throughout the body and the brain. Oxytocin receptors have been identified in the central nervous system, kidney, heart, thymus, pancreas, and adipocytes. Oxytocin is also produced in peripheral tissues, for example, the uterus, placenta, amnion, corpus luteum, and testis. The protein encoded by this gene belongs to the G-protein coupled receptor family and acts as a receptor for oxytocin. Its activity is mediated by G proteins which activate a phosphatidylinositol-calcium second messenger system.

A “transparent” or “clear” water soluble formulation, as disclosed herein, refers to a formulation that can be clearly seen through with the naked eye and is optionally colored.

Concentrations, amounts, solubility, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a numerical range of about 1 to about 4.5 should be interpreted to include not only the explicitly recited limits of 1 to about 4.5, but also to include individual numerals such as 2, 3, 4, and sub-ranges such as 1 to 3, 2 to 4, etc. The same principle applies to ranges reciting only one numerical value, such as “less than about 4.5,” which should be interpreted to include all of the above-recited values and ranges. Further, such an interpretation should apply regardless of the breadth of the range or the characteristic being described.

With this in mind, in the search for alternative mechanisms to treat mood disorders, it has been recognized that xanthohumol, a prenylflavonoid derived from the flowers of the hops plant, Humulus lupulus L., has great potential in the treatment of emotional imbalance, depression, mood disorders, and various other mental disturbances. These affective emotional states also relate to anti-social behavior and relationships between people such as married couples, family members, and others in a societal context. Furthermore, it has also been recognized that these types of compounds have the additional benefit of being suitable for treating certain skin disease or damage, such as inflammation disorders including eczema and atopic dermatitis, and can increase oxytocin production in the skin.

Furthermore, there is an increasing interest in the development of therapeutic compounds that may be used to improve focus, learning, memory and alertness, in both elderly and young people, individuals who need especially high memory and attention in their daily work, including students, construction workers, drivers, pilots, physicians, salespeople, executives, housewives, “high performance professionals” and people who are under mental or daily stress as well as persons who are prone to psychiatric instability and dramatic mood swings. A therapeutic product that can provide a balanced mental state would contribute to a more productive life for a large percentage of people who live a high stress, high performance life.

The oxytocic (OT) system seems to play a role in socioemotional functioning and positive emotion, and relates to empathy and stress reactivity in humans. OT has been shown to decrease cortisol levels, reduce the cardiovascular response to stress, and attenuate the amygdala's response to emotional stimuli. Because oxytocin is broadly involved in emotional and social processes, it is a potential target for the therapeutic treatment of various widespread mental disturbances related to everyday life in modern society. Oxytocin (OT) is a neurohypophysial peptide hormone (neuropeptide) that has been associated with an improvement in mood and emotional state, and modulation of the oxytocin receptors has potential to explain the positive action of the invention described herein. Recently OT expression and its receptor have been detected in human keratinocytes and dermal fibroblasts. Human experimental studies have indicated that an infusion of oxytocin, results in an increase in many aspects of pro-social behavior, trust, generosity, empathy, and sacrifice, all of which can be effected by stress. Therefore, it would be beneficial to provide a therapeutic product or drug that can stimulate endogenous production of oxytocin, and in particular, stimulate the genes related to the oxytocin receptor, or the transcription of genes that encode for oxytocin.

Additionally, it has been recognized that the formulations of the present disclosure are also useful for the treatment of skin disease, such as eczema, atopic dermatitis, and anti-aging skin care/repair or wrinkle reduction. OT decreases proliferation of dermal fibroblasts and keratinocytes in a dose-dependent manner. Oxytocin receptor (“OTR”) knockdown in dermal fibroblasts and keratinocytes leads to elevated levels of reactive oxygen species and reduced levels of glutathione (GSH). Moreover, OTR-depleted keratinocytes exhibited an increased release of the pro-inflammatory cytokines IL6, CCL5, and CXCL10. This suggests that the OT system modulates key processes which are dysregulated in atopic dermatitis (AD) such as proliferation, inflammation and oxidative stress responses. Furthermore, a downregulation of the OT system in peri-lesional and lesional atopic skin may also occur.

Thus, a compound or nutraceutical composition which may increase the concentration of oxytocin, enhance the affinity of oxytocin receptors or upregulates the expression of oxytocin receptors, thereby enabling improvements in learning, memory and alertness, enabling improved mood, would be desirable. Some or all of these advantages may be possible using the formulations of the present disclosure.

In accordance with this, a method for the treatment of mood disorders and/or skin disease or damage can comprise administering an extracted prenylflavonoid to a subject. Additional ingredients can also be included, such as a non-ionic surfactant and/or other additives or excipients. In one example, a stable, water-soluble pharmaceutical gel composition of prenylflavonoid is disclosed that can be prepared and administered as described herein. In this method, a water-soluble non-ionic surfactant is heated in a container to a temperature of about 90° F. to about 200° F. while mixing the non-ionic surfactant until a clear non-ionic surfactant is formed. An extracted prenylflavonoid is then added to the clear non-ionic surfactant and mixed until a clear non-ionic surfactant-prenylflavonoid combination is formed so as to constitute from about 70 wt % to 99.9 wt % surfactant, and from 0.01 wt % to 5 wt % prenylflavonoid, wherein the prenylflavonoid is sufficiently dispersed or dissolved in the surfactant so that a gel composition is formed containing no visible micelles or particles of prenylflavonoid. In administering this or other formulations to a subject, the resultant water-soluble pharmaceutical gel or concentrate can be administered, or it can be admixed with a liquid or solid carrier for administration.

The prenylflavonoid may be derived from a natural source, such as hops. Hops (Humulus lupulus L.) has been used for centuries as a bittering agent in the brewing of beer. Hops contain alpha acids such as humulone, co-humulone, ad-humulone, and beta acids such as lupulone and co-lupulone. Hops also contains many prenylflavonoids, such as xanthohumol, isoxanthohumol, desmethylxanthohumol, 8-prenylnaringenin, and 6-prenylnaringenin. Xanthohumol is a yellow-orange substance with a melting point of 172 degrees C. and a molecular weight of 354.4. A typical ethanol extract of hops yields about 3 mg/g (3%) of xanthohumol out of a total flavonoid content of 3.46 mg/g. Dried hop contains about 0.2 to 1.0% by weight xanthohumol. Xanthohumol can be extracted and purified to a concentration of greater than 90 wt %, in certain examples, to greater than 97 wt %, 98 wt %, or even 99 wt % pure. By extracting one or more of these prenylflavonoids, and formulating into a suitable food, supplement, beverage, or other medicinal dosage form, an effective formulation for treating mood disorders or eczema can be prepared.

Prenylflavonoids may be isolated from hops through purification, fractionation, or separation methods that are known to those skilled in the art. Ethanol may be used to extract higher levels of the prenylflavonoids from hops. The typical prenylflavonoid content of an ethanol extract of hops includes xanthohumol (3 mg/g), desmethylxanthohumol (0.34 mg/g), isoxanthohumol (0.052 mg/g), 6-prenylnaringenin (0.061 mg/g), and 8-prenylnaringenin 0.015 (mg/g). Supercritical carbon dioxide extractions tend to contain much lower levels, or non-existent levels of prenylflavonoids. In fact, these compounds are almost non-existent in standard CO₂ extracts because the prenylflavonoids are virtually insolvent on carbon dioxide. In the examples provided herein, a xanthohumol extract of purity of greater than 98 wt % has been used. It is noted herein that any method used to isolate prenylflavonoids are referred to herein as “extractions” or “extracted prenylflavonoids, regardless of how the isolation or concentration occurs.

Extracted prenylflavonoids that are useful as described herein can include prenylchalcones and/or prenylflavanones. In some embodiments, the prenylflavonoid is selected from xanthohumol, xanthogalenol, desmethylxanthohumol (2′,4′,6′,4-tetrahydrooxy-3-C-prenylchalcone), 2′,4′,6′,4-tetrahydrooxy-3′-C-geranylchalcone, dehydrocycloxanthohumol, dehydrocycloxanthohumol hydrate, 5′-prenylxanthohumol, tetrahydroxanthohumol, 4′-O-5′-C-diprenylxanthohumol, chalconaringenin, isoxanthohumol, 6-prenylnaringenin, 8-prenylnaringenin, 6,8-diprenylnaringenin, 4′,6′-dimethoxy-2′,4-dihydroxychalcone, 4′-O-methylxanthohumol, 6-geranylnaringenin, 8-geranylnaringenin, and metabolites and/or derivatives thereof. Prenylflavonoids of interest include xanthohumol, xanthohumol metabolites, and derivatives thereof, in extracted form. By extracting the prenylflavonoids and then using the extracted prenylflavonoids to prepare appropriate formulations, a pure form of these ingredients can be prepared and used at an appropriate concentration for treating these conditions.

In some embodiments, the xanthohumol can be present in the formulation at a concentration of at least 1%, 5%, 10%, 20%, 25%, 30%, 35%, 45%, 45%, or 50% by weight. In other embodiments the xanthohumol can be present in the pharmaceutical dosage form at a concentration from 0.01%, 0.1%, 1% to 80%, 5% to 50%, 10% to 35%, or 20% to 25% (by weight).

If a water-soluble gel or concentrate is to be formed (and then mixed with water as described herein), useful non-ionic surfactants include, for example, non-ionic water soluble mono-, di-, and tri-glycerides; non-ionic water soluble mono- and di-fatty acid esters of polyethylene glycol; non-ionic water soluble sorbitan fatty acid esters (e.g. sorbitan monooleates such as SPAN 80 and TWEEN 20 (polyoxyethylene 20 sorbitan monooleate)); polyglycolyzed glycerides; non-ionic water soluble triblock copolymers (e.g. poly(ethyleneoxide)/poly-(propyleneoxide)/poly(ethyleneoxide) triblock copolymers such as POLOXAMER 406 (PLURONIC F-127), and derivatives thereof.

Examples of non-ionic water soluble mono-, di-, and tri-glycerides include propylene glycol dicarpylate/dicaprate (e.g. MIGLYOL 840), medium chain mono- and diglycerides (e.g. CAPMUL and IMWITOR 72), medium-chain triglycerides (e.g. caprylic and capric triglycerides such as LAVRAFAC, MIGLYOL 810 or 812, CRODAMOL GTCC-PN, and SOFTISON 378), long chain monoglycerides (e.g. glyceryl monooleates such as PECEOL, and glyceryl monolinoleates such as MAISINE), polyoxyl castor oil (e.g. macrogolglycerol ricinoleate, macrogolglycerol hydroxystearate, macrogol cetostearyl ether), and derivatives thereof.

Non-ionic water soluble mono- and di-fatty acid esters of polyethylene glycol include d-α-tocopheryl polyethylene glycol 1000 succinate (TPGS), polyethylene glycol 660 12-hydroxystearate (SOLUTOL HS 15), polyoxyl oleate and stearate (e.g. PEG 400 monostearate and PEG 1750 monostearate), and derivatives thereof. Polyglycolyzed glycerides include polyoxyethylated oleic glycerides, polyoxyethylated linoleic glycerides, polyoxyethylated caprylic/capric glycerides, and derivatives thereof. Specific examples include LABRAFIL M-1944CS, LABRAFIL M-2125CS, LABRASOL, SOFTIGEN, and GELUCIRE.

In some embodiments, the non-ionic surfactant is a polyoxyl castor oil, or derivative thereof. Effective polyoxyl castor oils may be synthesized by reacting either castor oil or hydrogenated castor oil with varying amounts of ethylene oxide. Macrogolglycerol ricinoleate is a mixture of 83% relatively hydrophobic and 17% relatively hydrophilic components. The major component of the relatively hydrophobic portion is glycerol polyethylene glycol ricinoleate, and the major components of the relatively hydrophilic portion are polyethylene glycols and glycerol ethoxylates. Macrogolglycerol hydroxystearate is a mixture of approximately 75% relatively hydrophobic of which a major portion is glycerol polyethylene glycol 12-oxystearate.

When preparing a formulation of the extracted prenylflavonoid and non-ionic surfactant, typically, these two ingredients can be present at a weight ratio is from about 1:5 to about 1:200, though ratios outside of this range can also be used. Thus, in the water-soluble concentrate or gel, these two ingredients may be the only two ingredients presents, and the can be present within this ratio range. If admixed with a solid of liquid carrier and/or other excipients, this ratio can remain the same, such as when water is added to form a liquid beverage, or a solid carrier is added to form a tablet or capsule or food supplement.

For the preparation of consumable formulations from the pharmaceutical formulations, pharmaceutically acceptable carriers can be either solid or liquid. Solid form preparations include powders, tablets, bilayer tablets or capsules, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co, Easton Pa.

Suitable carriers include magnesium carbonate, magnesium stearate, talc, sugar, lactose, pectin, dextrin, starch (from corn, wheat, rice, potato, or other plants), gelatin, tragacanth, a low melting wax, cocoa butter, sucrose, mannitol, sorbitol, cellulose (such as methyl cellulose, hydroxypropylmethyl-cellulose, or sodium carboxymethylcellulose), and gums (including arabic and tragacanth), as well as proteins such as gelatin and collagen. If desired, disintegrating or co-solubilizing agents may be added, such as the cross-linked polyvinyl pyrrolidone, agar, alginic acid, or a salt thereof, such as sodium alginate. In powders, the carrier is a finely divided solid, which is in a mixture with the finely divided active component. In tablets, the active component is mixed with the carrier having the necessary binding properties in suitable proportions and compacted in the shape and size desired.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the invention can also be used orally using, for example, push-fit capsules made of gelatin, as well as soft, sealed capsules made of gelatin and a coating such as glycerol or sorbitol. Push-fit capsules can contain prenylflavonoid mixed with a filler or binders such as lactose or starches, lubricants such as talc or magnesium stearate, and, optionally, stabilizers. In soft capsules, the prenylflavonoid compounds may be dissolved or suspended in suitable liquids, such as fatty oils, liquid paraffin, or liquid polyethylene glycol with or without stabilizers.

After addition of the carrier to the pharmaceutical formulation, the consumable formulation may take the form of a water-soluble formulation, a beverage formulation, a food, formulation, a capsule formulation, a tablet formulation, an injectable formulation, a transdermal formulation, and any combination thereof.

In some embodiments, the water soluble formulation for administration can be a water solubilized formulation. A “water solubilized formulation,” as used herein, includes a prenylflavonoid such as xanthohumol, and a non-ionic surfactant, and water (e.g. a water containing liquid) but does not include organic solvents (e.g. ethanol). In some embodiments, the water solubilized formulation a transparent water soluble formulation.

In one aspect, the present disclosure provides a water-soluble formulation for administration that comprises or consists essentially of a prenylflavonoid, such as xanthohumol, and a non-ionic surfactant, and optionally water and/or excipients. In some embodiments, the water soluble formulation does not include a vegetable oil suspension or visible macro-micelles (micelles visible to the naked eye) in water. In other embodiments, the water soluble formulation does not include an alcohol (e.g. the compound is not first dissolved in alcohol and then added to water). In another aspect, the free form of the compound is preferred due to a higher concentration of the active compound.

In some embodiments, the water soluble formulation for administration includes a prenylflavonoid compound, or xanthohumol, and polyoxyl castor oil to form a transparent water soluble formulation. In certain embodiments, light may be transmitted through the transparent water soluble formulations without diffusion or scattering. Thus, in some embodiments, the transparent water soluble formulations are not opaque, cloudy or milky-white. Transparent water soluble formulations disclosed herein do not include milky-white emulsions or suspensions in vegetable oil such as corn oil. Transparent water soluble formulations are also typically not formed by first dissolving the compound in alcohol, and then mixed with water.

In some embodiments, a water soluble formulation for administration can be in the form of a pharmaceutical composition or beverage. The pharmaceutical composition may include a prenylflavonoid such as xanthohumol, or prenylflavonoid metabolite, a non-ionic surfactant, and a pharmaceutically acceptable excipient, or water. After a pharmaceutical composition including a prenylflavonoid of the invention has been formulated in an acceptable carrier, it can be placed in an appropriate container and labeled for treatment of an indicated condition. For administration of prenylflavonoids, such labeling would include, e.g., instructions concerning the amount, frequency and method of administration. In such embodiments, at least 0.5 mg, 1 mg, 2 mg, 3 mg, 4 mg, 5 mg, 10 mg, 20 mg, 30 mg, 40 mg, 50 mg, 100 mg, 200 mg, 300 mg, 400 mg, 500 mg, or 1 g of prenylflavonoid is present in the water soluble formulation. In other embodiments, 0.1 mg to 2 g, 0.5 mg to 1 g, 1 mg to 500 mg, 1 mg to 100 mg, 1 mg to 50 mg, 1 mg to 10 mg, or 1 mg to 5 mg of prenylflavonoid is present in the water soluble formulation. Any appropriate dosage form is useful for administration of the water soluble formulation of the present invention, such as oral, parenteral and topical dosage forms. Oral preparations include tablets, pills, powder, dragees, capsules (e.g. soft-gel capsules), liquids, lozenges, gels, syrups, slurries, beverages, suspensions, etc., suitable for ingestion by the patient. The formulations of the present invention can also be administered by injection, that is, intravenously, intramuscularly, intracutaneously, subcutaneously, intraduodenally, or intraperitoneally. Additionally, the formulations of the present invention can be administered transdermally. The formulations can also be administered by in intraocular, intravaginal, and intrarectal routes including suppositories. Thus, the formulations described herein may be adapted for oral administration.

In dietary compositions for administration, especially in food and beverages for humans, the prenylflavonoid or xanthohumol or any mixture of them, is suitably present in an amount in the range of from about 0.0001 (1 mg/kg) to about 5 weight-% (50 g/kg), preferably from about 0.001% (10 mg/kg) to about 1 weight-%, (10 g/kg) more preferably from about 0.01 (100 mg/kg) to about 0.5 weight-% (5 g/kg), based upon the total weight of the food or beverage. Beverages encompass non-alcoholic and alcoholic drinks as well as liquid preparations to be added to drinking water and liquid food. Non-alcoholic drinks are e.g. soft drinks, sport drinks, fruit juices, lemonades, near-water drinks (i.e. water-based drinks with low calorie content), teas and milk-based drinks. Liquid foods are e.g. soups and dairy products.

Xanthohumol may also be present, for example, in a tablet formulation, at a concentration from 0.5 to 50 mg per tablet. In other embodiments, the prenylflavonoid is present at a concentration from 0.01 mg/ml to 25 mg/tablet. The formulations may be administered as a unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The quantity of active component in a unit dose preparation may be varied or adjusted according to the particular application and the potency of the active component. The composition can, if desired, also contain other compatible therapeutic agents.

As mentioned, when preparing a suitable formulation, any formulation that effectively delivers the prenylflavonoid can be prepared. In one specific example, the prenylflavonoid can be prepared in a formulation by heating a water-soluble non-ionic surfactant in a container to a temperature of about 90° F. to about 200° F. while mixing the non-ionic surfactant until a clear non-ionic surfactant is formed; and adding a prenylflavonoid to the clear non-ionic surfactant and mixing until a clear non-ionic surfactant-prenylflavonoid combination is formed so as to constitute from about 70 wt % to 99.9 wt % surfactant and from 0.01 wt % to 5 wt % prenylflavonoid. The prenylflavonoid is thus sufficiently dispersed or dissolved in the surfactant so that a gel composition is formed containing no visible micelles or particles of dietary fatty acid. The solution may be heated to increase solubility. The heating temperature is typically selected to avoid chemical breakdown of the non-ionic surfactant. Water can be used to solvate the gel or concentrate to make a beverage in one example, or can be fortified directly (with or without added water) into soft gel capsules, creams, ointments, foods, etc.

In one specific embodiment, the temperature of both can be maintained at from 90 and 120° F. In some embodiments, the resulting solution is a water-soluble formulation or transparent water soluble formulation as described above. For example, the resulting solution may be a water soluble formulation that is a crystal clear solution, with no particles visible to the naked eye. Alternatively, the gel composition (prior to addition with water) will be combinable with warm water, as described above, to form a water soluble formulation. In another embodiment, the non-ionic surfactant is polyoxyl castor oil and the prenylflavonoid is xanthohumol.

In another aspect, the disclosure relates to the use of an effective amount of a prenylflavonoid, e.g., xanthohumol, or any mixture thereof, for the manufacture of a composition for the treatment of a disorder connected to mood or stress, such as depression, dysthymia, generalized anxiety disorder, cognitive dysfunction, impaired memory, mental fatigue, physical fatigue, emotional imbalance, occupational stress, insomnia, dysregulation of Circadian rhythm, and antisocial behavior. The invention relates to the use of an effective amount of a prenylflavonoid, xanthohumol, or any mixture thereof, particularly for the manufacture of an antidepressant, a mood/vitality improver, a stress reliever, a condition improver, a reducer of anxiety, a reducer of obsessive-compulsive behavior, a relaxant, a sleep improver and/or an insomnia alleviator and a cognitive enhancer. The disclosure also relates to the use of an effective amount of a prenylflavonoid, e.g., xanthohumol, or any mixture thereof, formulated with a non-ionic surfactant for the manufacture of a water soluble, or gastric soluble, composition for the treatment of a disorder connected to mood or stress, particularly for the manufacture of an antidepressant, a mood/vitality improver, a stress reliever, a condition improver, a reducer of anxiety, a reducer of obsessive-compulsive behavior, a relaxant, a sleep improver and/or an insomnia alleviator and a cognitive enhancer. Thus, the present invention is also directed to a method for the prevention of a mood disorder in animals including humans, said method comprising administering an effective dose of a prenylflavonoid, such as xanthohumol or any mixture thereof to animals including humans which are in need thereof. In this regard an effective dose of a prenylflavonoid and/or xanthohumol, may especially be used for maintaining the mental well-being, for maintaining a balanced cognitive function, for helping to reduce the risk of mood swings, for helping to retain a positive mood and for supporting cognitive wellness, and for helping to maintain a good sleep quality.

In another aspect, the disclosure relates to the use of an effective amount of a prenylflavonoid, e.g., xanthohumol, or any mixture thereof, for the manufacture of a composition for the treatment of skin disease or damage, for example, eczema, atopic dermatitis, or for improving the appearance of the skin, or to treat damaged skin.

The amount of xanthohumol sufficient to have a therapeutic effect on a subject with an affective mood disorder or skin disease or damage may be from about 0.5 mg to about 1000 mg, from about 1 mg to about 50 mg, from about 1 mg to about 20 mg, or about 3 mg to about 10 mg. In some embodiments, the dose of xanthohumol is 1 mg, 3 mg, 5 mg, 10 mg, or 20 mg. or 50 mg. In still other embodiments, the dose of xanthohumol is about 5 mg. The xanthohumol is typically administered as a twice per day formulation or as a once per day formulation.

In solid dosage unit preparations for humans, a prenylflavonoid or xanthohumol or any mixture of them, is suitably present in an amount in the range of from about 0.1 mg to about 1000 mg, preferably in the range of from about 1 mg to about 500 mg per dosage unit. More preferably, in a range of about 1 mg to about 100 mg. Dosages within these ranges can be relevant to both consumable compositions, as well as injectable or topical formulations, and can be modified within appropriate ranges as would be appreciated by one skilled in the art after considering the present disclosure.

In dietary compositions, especially in food and beverages for humans, the prenylflavonoid, e.g., xanthohumol, or any mixture of them, is suitably present in an amount in the range of from about 0.0001 (1 mg/kg) to about 5 weight-% (50 g/kg), preferably from about 0.001% (10 mg/kg) to about 1 weight-%, (10 g/kg) more preferably from about 0.01 (100 mg/kg) to about 0.5 weight-% (5 g/kg), based upon the total weight of the food or beverage. In food and drinks, the range is from 5 to 50 mg per serving, i.e. about 120 mg per kg food or drink.

For animals excluding humans a suitable daily dosage of a prenylflavonoid or xanthohumol, or any mixture of them, may be within the range of from 0.001 mg per kg body weight to about 1000 mg per kg body weight per day. More preferred is a daily dosage in the range of from about 0.1 mg to about 500 mg per kg body weight, and especially preferred is a daily dosage in the range of from about 1 mg to 100 mg per kg body weight.

A prenylflavonoid of interest, xanthohumol, may be incorporated into various types of dosage forms for convenient consumption by animals or humans. Examples of fortified foods are cereal bars and bakery items such as cakes and cookies. Additionally, the prenylflavonoid can be administered in the form of a beverage, food, feed, dairy product, yoghurt, fortified food, enhanced water, cereal bars, bakery item, cake, cookies, dietary supplement, tablet, pill, granules, dragees, capsules, effervescent formulations, non-alcoholic drinks, soft drinks, sport drinks, fruit juices, teas, milk-based drinks, liquid foods, soups, liquid dairy products, or any combination thereof.

Another embodiment can include observing a behavioral or autonomic effect of the prenylflavonoid on the patient and further modifying treatment based on the observed effect. Observed effects can include improved mood/vitality, relief of stress, reduced anxiety, reduced obsessive-compulsive behavior, relaxation, improved sleep, alleviated insomnia, enhanced of cognition, maintenance of cognitive wellness and balance, enhanced learning, enhanced language processing, enhanced problem solving, enhanced intellectual functioning, enhanced ability to cope with psychosocial burdens, enhanced attention and concentration, enhanced memory, enhanced mental alertness, enhanced mental vigilance, and stabilized mental status. In an embodiment using prenylflavonoid for the treatment of skin disease or damage, observed effect can include improved pruritis, decreased skin inflammation, and decreased blood levels of inflammatory cytokines.

The foregoing detailed description describes the disclosure with reference to specific exemplary embodiments. However, it will be appreciated that various modifications and changes can be made without departing from the scope of the present invention as set forth in the appended claims. The detailed description and accompanying drawings are to be regarded as merely illustrative, rather than as restrictive, and all such modifications or changes, if any, are intended to fall within the scope of the present invention as described and set forth herein.

### EXAMPLES

The prenylflavonoid used in the following Examples was extracted from hops flowers and was determined to be 98% xanthohumol by HPLC.

### Example 1

Water soluble compositions of xanthohumol were formulated containing the non-ionic surfactant macrogolglycerol hydroxystearate (polyoxyl 40 castor oil). The polyoxyl castor oil (non-ionic surfactant) was heated and stirred to a temperature of about 100° F., then, the powdered xanthohumol (98 wt %) was added slowly and mixed until a clear viscous solution was formed containing dissolved xanthohumol (hereinafter referred to as “the emulsion phase,” “gel,” or “water-soluble concentrate”). The emulsion phase formulation included macrogolglycerol hydroxystearate 40 and powdered xanthohumol. The xanthohumol/surfactant mixture was then slowly added to warm water (100° F.) until a crystal clear solution was formed.

### Example 2

Two ml of the water soluble liquid concentrate or gel formulation of Example 1 was added to 500 ml of water to make a pleasant tasting beverage drink or fortified water. 10 subjects suffering from mild depression and stress were instructed to consume a 500 ml bottle of this beverage once per day in the evening for two weeks, and were asked to fill out a daily questionnaire related to subjective feelings of improvement in mood, focus, emotional balance, and ability to sleep better. Eight of the ten subjects experienced a significant improvement in mood and restful sleep after 3 days of consuming the beverage. Two of the 10 subjects experienced a mild improvement, which varied from day to day, with some days experiencing a greater improvement than others. This could be related to other confounding issues related to diet, alcohol consumption, or other activities related to stress levels in their lives.

### Example 3

A pleasant tasting xanthohumol beverage is prepared in accordance with the formulation of Table 1. The beverage is clear in that no visible particles are detectable by the naked eye.

TABLE 1

| Ingredient | Wt % |
|---|---|
| Xanthohumol 98 wt % | 0.5% |
| Water | 89% |
| Polyoxyl Castor Oil | 10% |
| Sodium Benzoate | 0.06% |
| Potassium Sorbate | 0.04% |
| Citric Acid | 0.4% |
| Total | 100% |

### Example 4

A topical cream is prepared by admixing the following ingredients, as set forth in Table 2 below.

TABLE 2

| Ingredient | Wt % |
|---|---|
| Water | 40% |
| Prunus Amygdalus (Sweet Almond) Oil | 13% |
| Tocopheryl Succinate | 10% |
| Xanthohumol Hops Extract (98%) | 1% |
| Polyoxyl Castor Oil | 30% |
| Glycerol | 2% |
| Hydrogenated Lecethin | 1% |
| Cetearyl Alcohol | 3% |
| Total | 100% |

### Example 5

A topical cream is prepared by admixing the following ingredients, as set forth in Table 3 below.

TABLE 3

| Ingredient | Wt % |
|---|---|
| Water | 60.49% |
| Prunus Amygdalus (Sweet | 12% |
| Almond) Oil | |
| Tocopheryl Succinate | 10% |
| Cetearyl Alcohol | 2.8% |
| Petrolatum | 2% |
| Glycerin | 2% |
| Magnesium Aluminum Silicate | 1% |
| Butylene Glycol | 1% |
| Cyclopentasiloxane | 1% |
| Sorbitan Stearate | 1% |
| Ceteth-10 Phosphate | 0.6% |
| Dicetyl Phosphate | 0.6% |
| Hordeum Distichon (Barley) | 0.6% |
| Extract | |
| Phenoxyethanol | 0.52% |
| Hydrogenated Lecithin | 0.5% |
| Castor Oil | 0.5% |
| Steareth-2 | 0.5% |
| Caprylyl Glycol | 0.419% |
| Potassium Hydroxide | 0.35% |
| Lavandula Angustifolia | 0.3% |
| (Lavender) Oil | |
| Xanthohumol Hops Extract | 0.3% |
| (98%) | |
| Phellodendron Amurense Bark | 0.2% |
| Extract | |
| Santalum Album | 0.2% |
| (Sandalwood) Extract | |
| Xanthan Gum | 0.15% |
| Butyrosperum Parkii (Shea | 0.1% |
| Butter) | |
| Mangifera Indica (Mango) | 0.1% |
| Seed Butter | |
| Tetrasodium EDTA | 0.1% |
| Helianthus Annuus | 0.065% |
| (Sunflower) Seed Oil | |
| Sorbic Acid | 0.061% |
| Rosmarinus Officin | 0.035% |
| (Rosemary) Leaf Extract | |
| Grape Seed Extract | 0.01% |
| Total | 100% |

### Example 6

In an effort to understand the activity of xanthohumol on mood enhancement, a Gene Expression and DNA Microarray Study was conducted with xanthohumol to determine which genes might be up-regulated or down-regulated, and if any of these genes are associated with afflictive mental disorders, or certain neurohormones such as oxytocin.

DNA microarrays were used to screen xanthohumol for changes in the expression of thousands of different genes. DNA micro arrays are extremely powerful tools that allow users to analyze changes in gene expression by monitoring changes in the messenger ribonucleic acid (“mRNA”) of hundreds to thousands of genes in a single experiment. All cells function by using their genes to make protein products. This process starts by making an mRNA copy of the gene through a process called transcription. The mRNA copy is then translated into a protein that plays a functional role within the cell or the cell's environment. Since the process of gene expression is highly regulated, the amount of mRNA can be a good indicator of the level of activity for a specific gene. With the introduction of DNA microarrays, researchers can now rapidly obtain a much more global view of what is happening inside of a cell since the results of one or two array experiments can potentially generate data on changes in gene expression across the entire known human genome.

If a treatment does not exert an effect on a gene, then the median fluorescent intensity of the feature corresponding to that specific gene will be the same for both the scan from a red laser and the scan from a green laser. This would result in a ratio of 1. If a treatment increases the expression of a gene, then the median intensity of the feature would be greater in the scan from the red laser than the green, resulting in a ration that is greater than 1. A ratio that is less than 1 would, therefore, indicate that the treatment reduced the expression of a gene. This is called the “ratio of means.” In the present data, a ratio of greater than or equal to 1.3 as the cut-off for up-regulated genes and a ratio of less than or equal to 0.7 as the cut-off for down-regulated genes was used. These broader ranges are a more conservative judging criterion and account for possible sources of variation in fluorescence intensity that are not associated with the treatment.

By providing information on how active ingredients affect systems of immediate interest, the data from microarray experiments can provide a valuable library that catalogs in detail the effects of a specific material which can be reviewed at a future time. The array will also have data on enzymes involved in lipid metabolism, DNA repair enzymes which can be a factor in ageing, and antioxidant enzymes.

In addition to the information on active ingredients and raw materials that the DNA microarray can provide, the arrays can be used to better characterize the mechanisms of action related to certain therapeutic categories. The array data also allows the verification that a gene of interest is expressed in a certain model before running an experiment. The following tables provide the data based on the ratio of means after 500 μL of a xanthohumol liquid formulation with a concentration of 5 mg/ml xanthohumol was used in the following experiment. RNA was isolated from the tissues for subsequent analysis via DNA microarrays.

TABLE 4
Results of Full Thickness Tissue DNA Microarray Screening

| Name | Ratio of Means | Gene |
|---|---|---|
| NM-000915 | 2.488 | OXT ref NM_Homo sapiens, MRNA |
| NM_000916 | 3.5 | OXTR ref NM_Homo sapiens, MRNA |

TABLE 5
Up-regulation of Oxytocin Genes, Ratio of Means
(actual data used to calculate the ratio with the descriptions)

| Name | F635 Median | F532 Median | Ratio of Means | Gene |
|---|---|---|---|---|
| NM_000915 | 171 | 78 | 2.488 | OXT |
| NM_00916 | 53 | 43 | 3.5 | OXTR |

As described above, a gene is considered to be up-regulated if the ratio of means is above 1.3, and down-regulated if the ratio of means is below 0.7. As can be seen from the data, both the OXT and the OXTR genes were significantly up-regulated, with OXTR up-regulated by greater than 50%. The OXT gene is the Homo sapiens oxytocin/neurophysin I prepropeptide mRNA (NM_{—}000915), and the OXTR gene is the Homo sapiens oxytocin receptor mRNA (NM_{—}000916).

### Example 7

A beverage, such as that described above in Example 3, is administered to a subject experiencing a mood disorder, such as depression. After a week of administration of a 500 ml bottle of the xanthohumol beverage, the symptoms of eczema are decreased.

### Example 8

A beverage, such as that described above in Example 3, is administered to a subject experiencing stress and anxiety. After a few weeks of administration of a 500 ml bottle of the xanthohumol beverage, the symptoms of the mood disorders are alleviated.

### Example 9

A cream, such as that described above in Example 4, is administered topically to a subject experiencing a dermatitis outbreak. After a few days of thin application of the cream to the skin site, the symptoms of eczema are decreased.

### Example 10

A cream such as described in Example 5 is administered to the facial skin of subjects with damaged skin due to overexposure to sunlight (ultraviolet light). After 1 week, there is noticeable improvement in the appearance of the skin, and the skin is significantly softer and less rough in appearance.

What is claimed is:

1. A method of treating a mood disorder or a skin disease or skin damage, comprising administering a formulation comprising an extracted prenylflavonoid to a subject suffering from the mood disorder or the skin disease or skin damage.

2. The method of claim 1, wherein the prenylflavonoid is selected from a group comprising xanthohumol, xanthogalenol, desmethylxanthohumol (2′,4′,6′,4-tetrahydrooxy-3-C-prenylchalcone), 2′,4′,6′,4-tetrahydrooxy-3′-C-geranylchalcone, dehydro cycloxanthohumol, dehydrocycloxanthohumol hydrate, 5′-prenylxanthohumol, tetrahydroxanthohumol, 4′-O-5′-C-diprenylxanthohumol, chalconaringenin, isoxanthohumol, 6-prenylnaringenin, 8-prenylnaringenin, 6,8-diprenylnaringenin, 4′,6′-dimethoxy-2′,4-dihydroxychalcone, 4′-O-methylxanthohumol, 6-geranylnaringenin, 8-geranylnaringenin, their metabolites, their derivatives, and any combination thereof.

3. The method of claim 1, wherein the prenylflavonoid is xanthohumol.

4. The method of claim 3, wherein the xanthohumol is present at a concentration from about 0.01% to 50% by weight.

5. The method of claim 1, wherein the formulation further comprises a non-ionic surfactant.

6. The formulation of claim 5, wherein the non-ionic surfactant is selected from the group comprising non-ionic water soluble mono-, di-, or tri-glycerides; non-ionic water soluble mono- or di-fatty acid esters of polyethylene glycol; non-ionic water soluble sorbitan fatty acid esters; polyglycolyzed glycerides; non-ionic water soluble triblock copolymers; their derivatives; and combinations thereof.

7. (canceled)

8. The method of claim 5, wherein the non-ionic surfactant is polyoxyl castor oil.

9. The method of claim 5, wherein the extracted prenylflavonoid to non-ionic surfactant weight ratio is from about 1:5 to about 1:200.

10. The method of claim 1, wherein the formulation is in the form of a water-soluble concentrate or gel.

11-12. (canceled)

13. The method of claim 1, wherein the formulation further comprises a solid carrier suitable to form a consumable formulation and wherein the solid carrier is selected from a group consisting of magnesium carbonate, magnesium stearate, talc, sugar, lactose, pectin, dextrin, starch, gelatin, tragacanth, a low melting wax, cocoa butter, sucrose, mannitol, sorbitol, cellulose, gums, gelatin, collagen, and combinations thereof.

14. (canceled)

15. The method of claim 1, wherein the formulation further comprises a liquid carrier and wherein the liquid carrier is a consumable formulation that is a beverage formulation or the liquid carrier is suitable to form a topical or injectable formulation.

16. The method of claim 1, wherein the formulation further comprises a solid or liquid carrier to form a consumable formulation and wherein the consumable formulation comprises from about 0.5 mg to 1000 mg of the extracted prenylflavonoid.

17. The method of claim 1, wherein the formulation further comprises a solid or liquid carrier to form a consumable formulation and wherein the consumable formulation is in the form of a food.

18. The method of claim 1, wherein the formulation further comprises a solid or liquid carrier to form a consumable formulation and wherein the consumable formulation is a beverage formulation or a food formulation and the prenylflavonoid is present at a concentration from about 0.0001 wt % to about 5 wt %.

19. (canceled)

20. The method of claim 1, wherein the formulation further comprises a solid or liquid carrier suitable to form a consumable formulation and wherein the consumable formulation is in the form of a capsule or a tablet and wherein the prenylflavoniod is xanthohumol and the xanthohumol is present at a concentration from about 0.5 milligrams to about 50 milligrams per capsule or tablet.

21-22. (canceled)

23. The method of claim 1, wherein the extracted prenylflavonoid is provided from a Humulus lupulus L. plant, and the extracted prenylflavonoid is at least 90 wt % pure prior to admixing with other formulation ingredients.

24. The method of claim 23, wherein the extracted prenylflavonoid is xanthohumol.

25. The method of claim 23, wherein the extracted prenylflavonoid is at least 97 wt % pure prior to admixing with other formulation ingredients.

26. The method of claim 1, wherein the formulation consists essentially of the extracted prenylflavonoid, a non-ionic surfactant, water, and optional excipients.

27. The method of claim 1, wherein the subject is a member selected from the group consisting of a human, a pet, a companion animal, or a farm animal.

28. (canceled)

29. The method of claim 1, wherein the method is used to treat a mood disorder and wherein the mood disorder is selected from a group consisting of depression, dysthymia, generalized anxiety disorder, cognitive dysfunction, impaired memory, mental fatigue, physical fatigue, emotional imbalance, occupational stress, insomnia, dysregulation of Circadian rhythm, antisocial behavior, and combinations thereof.

30. The method of claim 29, wherein the prenylflavoniod is xanthohumal and the formulation is administered from once to twice per day.

31-33. (canceled)

34. The method of claim 29, wherein the prenylflavonoid is administered at a dose from about 0.1 milligram to 1000 milligrams.

35. The method of claim 29, wherein the prenylflavonoid is administered at a dose from about 0.001 mg/kg body weight to 1000 mg/kg body weight.

36. (canceled)

37. The method of claim 29, further comprising the step of observing a behavioral or autonomic effect of the prenylflavonoid on the subject and further modifying treatment based on said effect, wherein the effect is selected from a group consisting of improved mood or vitality, relief of stress, reduced anxiety, reduced obsessive-compulsive behavior, relaxation, improved sleep, alleviated insomnia, enhanced of cognition, maintenance of cognitive wellness and balance, enhanced learning, enhanced language processing, enhanced problem solving, enhanced intellectual functioning, enhanced ability to cope with psychosocial burdens, enhanced attention and concentration, enhanced memory, enhanced mental alertness, enhanced mental vigilance, stabilized mental status, and combinations thereof.

38. The method of claim 37, wherein the observed effects include both behavioral and autonomic effects.

39. The method of claim 29, wherein administering the prenylflavonoid increases oxytocin gene expression in the subject.

40. (canceled)

41. The method of claim 1, wherein the method is used treat the skin disease or skin damage, and comprises administering a formulation of an extracted prenylflavonoid to a subject suffering from the skin disease or skin damage.

42-68. (canceled)

69. The method of claim 41, wherein the skin disease or skin damage is selected from the group consisting of eczema, atopic dermatitis, or is related to aging or wrinkles.

70-71. (canceled)

72. The method of claim 41, wherein the prenylflavonoid is xanthohumol and the formulation is administered from once to twice per day.

73-77. (canceled)

78. The method of claim 41, wherein the prenylflavonoid is administered at a dose from about 0.1 milligram to 1000 milligrams.

79. The method of claim 41, wherein the prenylflavonoid is administered at a dose from about 0.001 mg/kg body weight to 1000 mg/kg body weight.

80. (canceled)

81. The method of claim 1, wherein the step of administering the prenylflavonoid increases oxytocin production in skin of the subject.
